(19) United States
(12) Patent Application Publication
(10) Pub. No.: US 2016/0279432 A1
(43) Pub. Date: Sep. 29, 2016

(54) SYSTEM FOR PACEMAKER PATIENTS AND THEIR CARDIOLOGISTS TO COMMUNICATE WITH ONE ANOTHER AND METHOD OF USE

(71) Applicant: Phillip S. Chappel, Washington, PA (US)

(72) Inventor: Phillip S. Chappel, Washington, PA (US)

(21) Appl. No.: 15/063,025

(22) Filed: Mar. 7, 2016

Related U.S. Application Data
(60) Provisional application No. 62/128,859, filed on Mar. 5, 2015.

(51) Int. Cl. A61N 1/372; A61N 1/37; A61B 5/00; A61B 5/11; A61B 5/0205

(52) U.S. Cl. CPC A61N 1/37282; A61B 5/1118; A61B 5/02055; A61B 5/0022; A61B 5/1112; A61N 1/3702; A61B 5/024

(57) ABSTRACT

A system for a pacemaker patient and his/her cardiologist to stay in near constant communication with one another comprises: (a) a database securely stored on a cloud server with specific log on criteria for the patient and the cardiologist to both fully access, said database including information particular to a pacemaker unit previously installed in the patient and: up-to-date activity tracking data for the patient; periodically-measured health vitals for the patient including heart rate, breathing rate and skin temperature when awake and when sleeping; and (b) a fitness monitoring band that the patient wears for tracking activities performed by the patient and for recording and transmitting to the database the periodically-measured health vitals for the patient. The system further includes at least one means for the cardiologist to alert the patient should an irregular measurement in health vitals be detected with the monitoring band. A method for using the system is also disclosed.

### CROSS-REFERENCE TO RELATED APPLICATION

This application is a perfection of Provisional Application Ser. No. 62/128,859, filed on Mar. 5, 2015, the disclosure of which is fully incorporated by reference herein.

### BACKGROUND OF THE INVENTION

1. Field of the Invention

This invention relates to a system that employs an improved or customized personal care monitoring device, sometimes referred to as a FITBIT®, and/or its competitive equivalents. More particularly, it relates to a system (and related method of use) that allows an individual wearer who already has an implanted pacemaker device to stay in close, near constant communication with his/her cardiologist/physician. The device may be marketed under the proposed trademark: Cardiac Care Communicator or C3.

2. Relevant Art

There are numerous website disclosures of fitness care wristband products, some of which are patented or patent pending. Many fitness monitoring devices include warnings for patients who have previously had pacemakers surgically installed. The inventor is one such past pacemaker patient/recipient. One such model is the Nike Fuelband as can be seen at: http://www.nike.com/us/en_us/c/nikeplus-fuel.

Recently, Medtronic received FDA clearance for a mobile app that allows patients to remotely forward data from their pacemakers to their physicians. The app, paired with a device called the MyCareLink Smart Monitor, reads data from the pacemaker and transmits it via Bluetooth to the patient's personal smartphone or tablet. But it is purely a one-way pathway. The patient cannot access his/her own data transmissions.

### SUMMARY OF THE INVENTION

This invention is a significant improvement over known fitness monitors and/or the aforementioned Medtronic phone app. While it also “monitors” a patient's personal pacemaker, and related day-to-day activities, it does so essentially with the help of, and in total “live” communication with, that patient's personal cardiologist. The link between patient and cardiologist is the major thing that this invention provides and, needless to say, it will be safe to wear for people with pacemakers.

The primary function of the C3 is to create a solid, ongoing communication link between each device-wearing patient who has a pacemaker and his/her cardiologist. It will have a number of similar functions that already exist with a number of activity bands but further communicate back and forth, between both parties in a live linking relationship. As such, this invention provides a need via its system and its method of use.

### DESCRIPTION OF PREFERRED EMBODIMENTS

In the physical application of this system, a patient's cardiologist can download the information to view that patient's C3 via a secure website using a specific pin which is e-mailed to the cardiologist by way of the secure network. Alternately, the cardiologist may install software on his/her computers via a disc mailed to the health provider's office. With a specially assigned PIN (identifier), the cardiologist can enter each patient's personal information concerning his/her Pacemaker model, installation date, service record and/or proposed replacement date (i.e., when to start one's replacement/upgrade protocol). Still other vital patient information such as his/her name, address, height, weight, and healthcare insurer are also inputted. Preferably, once entered, the cardiologist will not be able to adjust the patient-provided additional access information.

The patient/customer who then joins the C3 monitoring system, visits the secure website and creates a personal data account. There, the patient enters a username of his/her choice, an e-mail address, cell phone link (for receiving/sending texts) and selects a secure password. A link will be available should the patient/user forget his/her username and/or password. There will also be password reminder question(s). On a supplemental (or alternative) basis, the patient/customer may also log in via his/her own, secure Facebook account.

The patient will next input his/her own personal information that can be specifically accessed by his/her cardiologist via the aforementioned upload link. Once fully entered from the initial feed, that information will be securely uploaded to the Cloud for VERY limited access by only the patient and his/her specially assigned cardiologist. The patient is then ready to begin wearing/using his/her own personalized Cardiac Care Communicator band.

The Cardiac Care Communicator band will have its own USB cable for attaching to the patient's own personal computer for: downloading, updating and periodically recharging the band. Once the USB has been linked to the computer's USB port, the patient/customer can log on with secured internet access and his/her personalized username/password will be entered.

As for activities/exercise monitoring, the patient can adjust his/her own goals and make other random adjustments that will better inform the cardiologist of any changes to their own health status. There will also be a NOTE SECTION where they can inform their cardiologist of any issues AND ask any questions pertaining to their pacemaker device that they may have or may not be able to find online.

With the C3 system, both patient and cardiologist can monitor, in real time activity items like:

Patient runs, distance walked per a given time period, stair tracking, bicycle rides and other exercise activities such as swimming, sports playing (tennis and golf).

It will also track the patient's light and deep sleep patterns. It will include a built-in heart monitor and track skin temperatures. Ideally, it can also provide each patient with food logging capabilities via a separately downloadable application.

The system should be waterproof for at least 50 meters depth, made from allergy resistant metals and/or composites, while further functioning as: a stopwatch, calorie counter; GPS device and/or pace tracker. It should have audible beeps for the patient to hear, as needed. But most of all, the system will provide rich data analysis and professional medical evaluations for both patient AND cardiologist to access via the C3 website with fresh and up-to-date synchronizations.

Each C3 band should further include: a heart-health indicator light, a vibration alert for when certain heart rate levels are reached, alarms for any and all pacemaker irregularities observed and an LED display. More advanced models may further include caller ID and text messaging for phones connected to any such band/device. (However, it may not be able to respond back to texts via the C3). Still other options include a remote control for playing music.

Additonal features on each C3 model will include a Vibrate alert so that the cardiologist may “buzz” the patient to send upcoming appointment reminders AND to immediately notify the patient should any serious issues arise pertaining to his/her pacemaker model, and its mainteance and care.

Finally, the C3 band should be able to provide its wearer/patient with periodic movement alerts so as to advice when it is time for him or her to next “move”. Such alerts can be changed by either the patient OR the cardiologist through a simple C3 website log on. The bands will not requre a smart phone connection but the latter may be enabled to add still other C3 features to the system as a whole.

The chief aspect that separates the C3 from all competition in the activity band market is the fact that it is specifically meant to serve as a supplement to any one patient's pacemaker.

It is to be understood that the present invention is not limited to the foregoing particulars. Other modifications and variations are possible as appreciated by those skilled in the art in light of the foregoing.

What is claimed is:

1. A system for a pacemaker patient and a cardiologist seen by the patient for both the patient and the cardiologist to stay in near constant communication with one another, said system comprising:
(a) a database securely stored on a cloud server with specific log on criteria for the patient and the cardiologist to both fully access, said database including information particular to a pacemaker unit previously installed in the patient and:
(i) up-to-date activity tracking data for the patient;
(ii) periodically-measured health vitals for the patient including heart rate, breathing rate and skin temperature when awake and when sleeping; and
(b) a fitness monitoring band that the patient wears for tracking activities performed by the patient and for recording and transmitting to the database the periodically-measured health vitals for the patient,
said system including at least one means for the cardiologist to alert the patient should an irregular measurement in health vitals be detected with the monitoring band.

2. The system of claim 1 wherein the database includes means for the cardiologist to remind the patient of upcoming medical appointments.

3. The system of claim 1, which includes means for notifying the patient of any performance irregularities with respect to the particular pacemaker installed in the patient.

4. The system of claim 1, which includes means for notifying the patient of any performance issues, including servicing recalls, from a manufacturer of the model of pacemaker installed in the patient.

5. The system of claim 1, which includes means for notifying the patient when a next pacemaker installation protocol should be commenced.

6. The system of claim 1 wherein the fitness monitoring band tracks activities performed by the patient, said activities selected from the group consisting of walking, running, stair tracking, swimming, sports playing (tennis and golf) and bicycling.

7. The system of claim 1, which further includes a feature for logging in what foods have been consumed by the patient, such as a calorie counter.

8. The system of claim 1, which further includes a GPS device.

9. The system of claim 1, which further includes an ability to play music for the patient to hear while exercising.

10. A method for enabling a pacemaker patient and a cardiologist seen by the patient to stay in near constant communication with one another, said method comprising:
(a) providing a database securely stored on a cloud server with specific log on criteria for the patient and the cardiologist to both fully access, said database including information particular to a pacemaker unit previously installed in the patient and:
(i) up-to-date activity tracking data for the patient;
(ii) periodically-measured health vitals for the patient including heart rate, breathing rate and skin temperature when awake and when sleeping;
(b) providing a fitness monitoring band that the patient wears for tracking activities performed by the patient and for recording and transmitting to the database the periodically-measured health vitals for the patient,
said method allowing both the patient and the cardiologist to fully access the database, makes changes to a profile for the patient stored and periodically updated on the database and further allowing the cardiologist to alert the patient should an irregular measurement in health vitals be detected with the monitoring band.

11. The method of claim 10 wherein the database includes means for the cardiologist to remind the patient of upcoming medical appointments.

12. The method of claim 10 wherein the monitoring band includes means for notifying the patient of any performance irregularities with respect to the particular pacemaker installed in the patient

13. The method of claim 10 wherein the monitoring band includes means for notifying the patient of any performance issues, including servicing recalls, from a manufacturer of the model of pacemaker installed in the patient.

14. The method of claim 10 wherein the monitoring band includes means for notifying the patient when a next pacemaker installation protocol should be commenced.

15. The method of claim 10 wherein the fitness monitoring band tracks activities performed by the patient, said activities selected from the group consisting of walking, running, stair tracking, swimming, sports playing (tennis and golf) and bicycling.

16. The method of claim 10 wherein the fitness monitoring band further includes a feature for logging in what foods have been consumed by the patient, such as a calorie counter.

17. The method of claim 10 wherein the fitness monitoring band further includes a GPS device.

18. The method of claim 10 wherein the fitness monitoring band further includes an ability to play music for the patient to hear while exercising.
